(19) United States
(12) Patent Application Publication
(10) Pub. No.: US 2011/0177178 A1
(43) Pub. Date: Jul. 21, 2011

(54) Extracts of Deschampsia antarctica Desv. with antineoplastic activity

(75) Inventors: Manuel Gidekel, Santiago (CL); Helga Weber, Pucon (CL); Gustavo Cabrera, Temuco (CL); Ana Gutierrez, Temuco (CL); Jennifer Osorio, Temuco (CL); Osvaldo Podhajcer, Buenos Aires (AR); Eduardo Cafferata, Buenos Aires (AR); Carlos Sunkel, Madrid (ES); Ivan Mihovilovic, Miami, FL (US)

(73) Assignee: Uxmal S.A., Chile, Santiago (CL)

(21) Appl. No.: 12/928,317

(22) Filed: Dec. 8, 2010

Related U.S. Application Data
(63) Continuation-in-part of application No. 12/734,597, filed on May 11, 2010.
(60) Provisional application No. 61/003,058, filed on Nov. 14, 2007.

(30) Foreign Application Priority Data
Jul. 14, 2010 (CL) CHILE 00753

(51) Int. Cl. A61K 36/899; A61P 35/00

(52) U.S. Cl. 424/750

(57) ABSTRACT

This invention describes antineoplastic compound derived from Deschampsia antarctica plants. The biologically active compounds are further characterized. The described compounds are demonstrated to have a capacity to inhibit cancer cell proliferation.

### PRIORITY

This is a continuation-in-part application of application Ser. No. 12/734,597 and this application claims priority of Chilean national application number 00753 filed on Jul. 14, 2010.

### FIELD OF INVENTION

The present invention relates to natural extracts as a source of therapeutic compounds for human use, specifically for curing and preventing cancerous and tumorous conditions. More specifically, the present invention relates to extracts, compositions or the extracts and methods to produce the extracts from Deschampsia antarctica for prevention of cancer.

### DESCRIPTION OF THE INVENTION

Cancer is the second leading cause of death in developed countries. Due to the parallel increase in the incidence of this disease as compared to the increase in the life expectancy of the population, there is a great medical and social interest in this pathology. The five types of cancer most common in the world are lung cancer, stomach cancer, breast cancer, colorectal cancer and uterine cancer.

The incidence and mortality from cancer have increased in many countries. According to the World Health Organization (WHO), more than 10 million people suffer cancer each year. That number is expected to increase by 2.4% annually, to 15 million people per years by 2010.

In the specific case of colorectal cancer (CRC), each year approximately one million new cases occur in the world and half a million deaths, the world mortality rate being 8.2/100,000 inhabitants. This type of cancer is largely seen in the more developed regions (25.1/100,000 inhabitants), representing the second leading cause of death from cancer in Europe and in the United States. More than 130,000 new cases are diagnosed annually in the United States alone and more than 370,000 new cases in Europe. In Argentina, the figures for new cases are similar to those occurring in the United States.

In Chile, cancer is the second leading cause of death. Intestinal cancer is responsible for 46.2% of the deaths from this pathology. Among that number, colorectal cancer is the third leading cause of death and it is constantly increasing in frequency. The mortality from this disease presented a significant rising trend in the period 1999-2003.

Close to 70% of patients who suffer from colorectal carcinoma must undergo a surgical resection while 30% to 40% have a relapse. The liver is the most frequent site of CRC metastasis and a complete resection of the hepatic metastasis is the only alternative cure. However, surgery is possible for only 20% of patients at the time of diagnosis and the average survival rate at 5 years is from 25% to 40%, even with chemotherapy.

The actual CRC treatment is ineffective in the advanced stages of the disease. An average survival rate of patients with metastatic colorectal carcinoma who receive state-of-the-art medication, like Erbitux and Avastin, as part of the first line of treatment is only 15 to 20.5 months. This demonstrates the need to look for more effective therapies to increase the patient's chances of survival.

On the other hand, colonoscopy is the only screening that allows the detection and removal of pre-malignant lesions. However, the test is very complex, in terms of patient preparation and inconvenience and patient discomfort. Therefore, CRC diagnosis at a relatively early stage is a rare event and only 9% of patients are detected state 1 of the disease when the possibilities of cure are larger. Therefore, the possibility of preventing CRC development by using nutraceutical compounds is of high importance.

This invention comprises natural extracts containing compounds with antineoplastic activity that will help to cure and prevent diseases that have a high incidence among the population. In particular, a natural product is described, said product being extracted from an organism adapted to survive the high radiation on the Antarctic Continent.

On the other hand, Deschampsia antarctica Desv. Is one of the two phanerogamous plants that have successfully colonized the Antarctic. It is found on several ocean islands in the south and it is restricted in the Antarctic territory to the Antarctic Peninsula. And island offshore. This taxon has adapted to survive in hostile conditions. Among the characteristics that might be involved in the survival of this grass in the adverse Antarctic environment are a tolerance to extracellular ice, a photosynthetic apparatus that maintains 30% of the photosynthetic optimum at 0° C., and the accumulation of carbohydrates as fructans and saccharose. The Antarctic territory is an adverse zone for the development of vascular plants. D. antarctica only grows in the summer season when photosynthetic active radiation (PAR) can reach 2000 μmol m⁻²s⁻¹ and the temperature is usually from −2 to 5° C.

Research on natural products as a source of therapeutic compounds for human use reached its peak in the 1980's. 49% of the 877 small molecules introduced on the market between 1981 and 2002 came from natural products or their synthetic derivatives (Newman, D J; J. Nat. Prod. 66: 1002-1037 (2003)). Despite this, pharmaceutical research on natural products has experienced a slight drop in the last decade. This was essentially due to the search for new compounds based on the development of combinatorial libraries and progress in molecular biology that have led to the design of “smart” chemical molecules like Iressa/Imanitib, aimed to target Epidermal Growth Factor Receptors. However, the experience in recent years has revived the interest of pharmaceutical companies in compounds derived from natural products, since it has been demonstrated that they have a greater number of chiral centers and steric complexity than any synthetic product or recombinant library (Free M, J. Chem. Inf. Comput. Sci. 43:218-227 (2003)). The main reason for this failure is that combinatorial libraries essentially look for compounds based on chemical accessibility but they have restrictions in terms of increasing chemical diversity (Martin Y C, J. Comb. Chem. 1:32-45 (1999)).

There are publications suggesting beneficial effects of fruit- and vegetable consumption in lowering the risk of various cancers, including colorectal cancer. Kaur et al. provide data showing chemo preventive effects of grape seed fruits (Kaur M, Clin. Cancer Res. 12(20): 6194-6202 (2006)). Lu et al. show data suggesting chemo preventive effects of green tea on lung cancer (Lu Y, Cancer Res. 66 (4): 1956-1963 (2006)). Suggestions of natural ingredients having chemo preventive effects along with the failure to meet the expectations regarding combinatorial chemistry have revived a pharmaceutical interest in natural compounds.

Accordingly, there is a constant need for natural compounds and extracts that could help preventing and curing the commonly occurring cancer diseases.

### SUMMARY OF THE INVENTION

This invention comprises natural extracts containing compounds with antineoplastic activity that will help to cure and prevent diseases that have a high incidence among the population. In particular, a natural product is described, said product being extracted from and organism adapted to survive the high radiation on the Antarctic Continent.

This invention provides a novel antineoplastic extract and a method to obtain the extract from Deschampsia antarctica.

This invention provides a natural antineoplastic extract to prevent proliferation of cancer cells.

Furthermore, this invention provides active composites of the natural antineoplastic extract.

This invention also provides compositions for treating patients with existing cancer conditions.

Moreover, this invention provides a method to increase the amount of antineoplastic compounds in Deschampsia antarctica tissue, and accordingly a method to extract the compounds of the plant tissue.

Even further, this invention provides a method to obtain antineoplastic extract from in vitro grown Deschampsia antarctica plants.

Still further, this invention provides a method to prepare in vitro cultivated Deschampsia plants for material of antineoplastic preparations.

Accordingly, this invention also provides antineoplastic preparations comprising Deschampsia antarctica extract or components of the extract.

Even further this invention characterizes components of the extract and discloses components previously unknown to be present in Deschampsia plants.

### BRIEF DESCRIPTION OF THE FIGURES

FIG. 1. UV-Vis 200-400 nm spectra diagram. A) Extract from plants of Deschampsia antarctica collected in vivo. B) Extract from plants of Deschampsia antarctica cultivated in vitro without treatment (Control).

FIG. 2. Effects of fractions of Deschampsia antarctica in concentrations of 100 μg/ml on in vitro growth of colon cancer cells (HT29 and LoVo), hepatic cancer cells (Hep3B) and control cells (wi38). GCB1, GCB2 and GCB3 correspond to extraction of three different collections of Deschampsia antarctica plants from the Antarctica. The extracts were prepared with water, then dried and dissolved in methanol. Control contains no extract added and the methanol control is a control containing 5% methanol as was used to dissolve the dried aqueous extract.

FIG. 3. Effects of ethyl acetate (a and b) and methanol (c and d) extracts at to concentration of 75 ug/ml on LoVo colorectal cancer cells (a and c) and we38 human fetal fibroblast cells (b and d).

FIG. 4. Effects of luteolin derivatives (peak 1 and peak 2) obtained from an extract of Deschampsia antaractica plants at different concentrations (0.017, 0.17 and 1.7 mM) on cellular viability of colorectal cancer cells LoVo. a) effects of Peak 2 derivative and b) effect of a combination of peak 1 and peak 2 derivatives.

FIG. 5. HPLC chromatogram of the main compounds of Deschampsia antarctica extracts. A) peak 1 and B) peak 2.

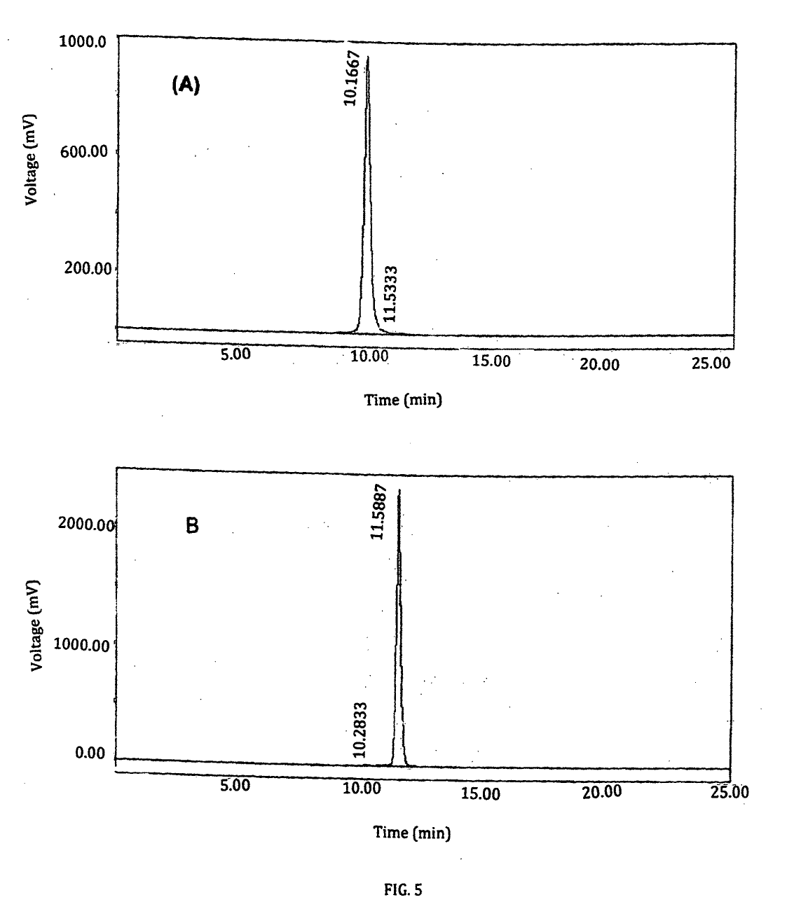

FIG. 6. UV-visible spectra of the main compounds of Deschampsia antarctica extracts. a) peak 1 spectrum and b) speak 2 spectrum.

FIG. 7. Mass spectra of a) peak 1, and b) peak 2 isolated from Deschampsia antarctica extract.

FIG. 8. Chemical structure of A) peak 1 compound and B) peak 2 compound.

FIG. 9. Chemical structure of orientin.

FIG. 10. Liquid chromatogram of the aqueous extract from Deschampsia antartica

FIG. 11. Total flavonoid content in peaks 6 and 10 (of chromatogram in FIG. 10) in dried samples collected in 2008 (dark column) and 2009 (light column) Antarctic campaign.

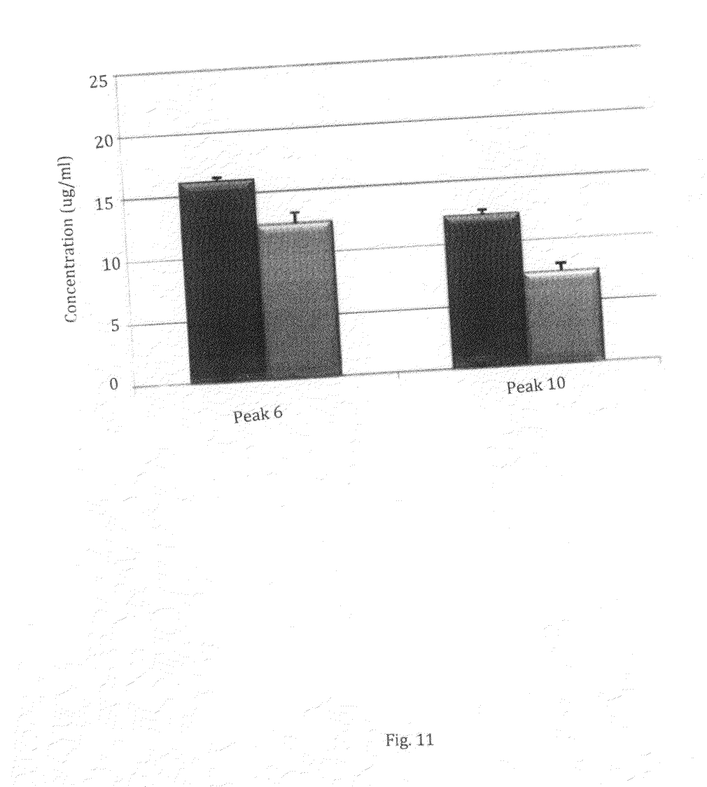

FIG. 12. Antineoplastic activity of individual peaks (of chromatogram in FIG. 10) at two different concentrations using a colon tumoral cells (Lovo).

FIG. 13. Antineoplastic activity of peak 6 and 10 (of chromatogram in FIG. 10) at different concentrations on colon tumoral cells (Lovo).

FIG. 14. Antineoplastic activity of peak combinations at different concentrations. The combination of peaks is indicated on top of the columns; e.g. 10-11 is a fraction that includes both peak 10 and peak 11 compounds.

### DETAILED DESCRIPTION OF THE INVENTION

Deschampsia antarctica Desv. (Poacea) is one of the two vascular plant species that have naturally colonized Maritime Antarctic Peninsula. During the recent years, D. antarctica has experienced an increasing exposure to ultraviolet radiation, due fundamentally to the hole in the ozone layer present in the Antarctic Region. Consequently, this plant species has modified its metabolism to increase the production of secondary metabolites that intervene in the photo protection process.

The fact that D. antarctica is naturally acclimated to conditions that expose it to oxidative stress (high light and low temperature) let us to consider this plant as a source of antioxidative compounds. It was possible to obtain antioxidative compounds from the plants that grew in wild in Antarctica but the feature was practically lost when the plants were cultivated in vitro. This disclosure provides methods to induce antioxidative compounds in in vitro grown plants and further more the invention according to this disclosed provides an antineoplastic extract obtained from the plants.

This invention authentically establishes the antitumorigenic effect of the extracts obtained from D. antarctica and their capacity to prevent the disease through the study of their in vitro effects.

This invention also describes a method to induce antineoplastic compounds in in vitro grown plants and isolation of the compounds, as well as their potential applications.

The products according to this invention are based on the metabolites with antineoplastic activity present in D. antarctica.

The invention is described below by means of examples. The examples are not meant to limit the scope of the invention.

### Example 1

### Comparison of the Absorption Peaks of Extract from Naturally Grown Plants to the Extract of In Vitro Grown Plants without Stress Induction

Deschampsia antarctica material was collected from Robert Island, a copper mine peninsula (62°22′S; 59°43′W), and it was carried in plastic bags. The material was disinfected with fungicide (Benomyl and Captan) and sodium hypochlorite. Plant material was micropropagated in vitro. The culture medium was prepared based on the Murashige and Skoog (MS) medium. 1 mg/l of BAP hormone (N6 Benzylaminopurine) was added as well as 35 mg/l saccharose and 9 g/l of agar at a final pH of 5.7. The in vitro growing plants were kept in growth chambers at 22° C. with a photoperiod of 16/8 h (light/darkness) and a photon flow of 2000 μmol m⁻² s⁻¹.

The aerial parts of the in vivo or in vitro plants were collected and macerated in 5 ml of distilled water. The maceration is later sonicated for 10 minutes and centrifuged at 1,000 rpm for 15 minutes

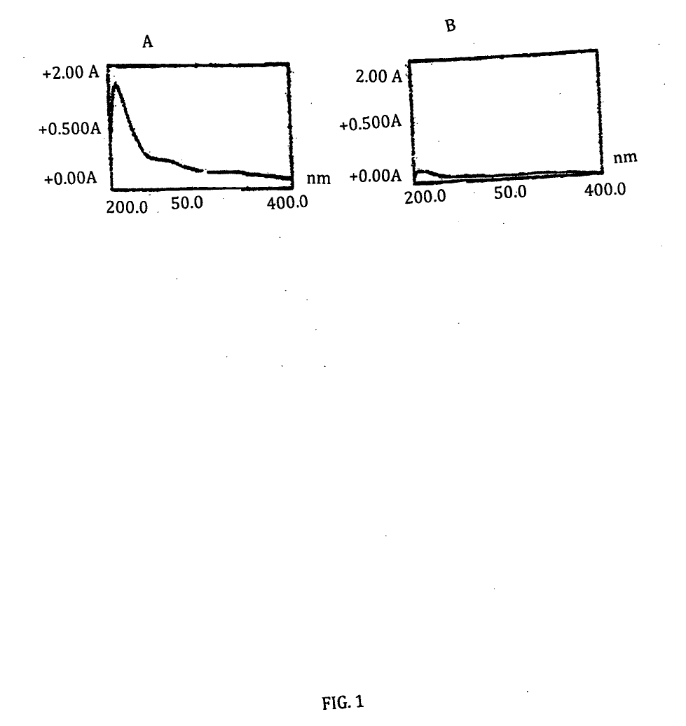

A thin layer chromatography was performed. The extracts were seeded on a 60 F₂₅₄ silica gel slide (MERCK) to visualize the compounds present. A UV-Vis SHIMADZU UV-160 spectrophotometric analysis was used to analyze the extracts. An absorbency screening was consequently conducted between 200 and 400 nm to determine the presence of absorption maximums characteristic of the families of compounds present in the extracts (FIG. 1).

When the flavonoids are dissolved in methanol, flavones and flavonols exhibit two major peaks of absorption in the 240-400 nm region when they are examined by UV spectroscopy and visible light. These peaks commonly refere to band I (300-400 nm) and band II (240-280 nm). According to the UV-Visible 200-400 nm spectrum analysis (FIG. 1), flavonoids are seen in the metabolic extracts of plants collected in vivo in the Antarctic. They are virtually absent in the extracts of plants cultivated in vitro in the laboratory. The flavonoids exhibit characteristic peaks.

The polyphenol content of the extract of plants grown in the Antarctic was 4.3% (w/w) and in vitro grown plants the content was 3.6% (w/w).

### Example 2

### Deschampsia Extracts in Various Solvents

In order to determine which solvent was better for Deschampsia antarctica metabolites extraction several solvents was employed. In table 1 the amount of extract obtained from 30 g of plant leave tissue was presented as well as the extraction yield (w/w %). The consecutive extraction with hexane and then ethyl acetate (EA) was represented as hexane→EA. Each extraction procedure was repeated three times in independent assay for each solvent and result expressed as the Mean±SD (Table 1).

TABLE 1
Extraction of metabolites from Deschampsia antarctica
leaf- tissue using different solvents.

| | Extract weight* | Extraction yield** |
|---|---|---|
| Solvent | (g) | (wt-%) |
| Water | 2.36 ± 0.05 | 36.8 ± 0.4 |
| Hexane | 0.13 ± 0.01 | 2.1 ± 0.2 |
| Ethyl acetate (EA) | 0.43 ± 0.02 | 6.9 ± 0.4 |
| Methanol | 1.29 ± 0.06 | 20.8 ± 1.0 |
| Hexane→EA | 0.08 ± 0.01 | 1.4 ± 0.1 |

*Calculated from fresh matter.
**Calculated from dried matter.

The extraction yield analysis indicates that yield parameter increases along with the solvent polarity, water and methanol being the better solvent systems. However, due to the higher potential of scaling up the process water was selected as the solvent for further preparation of Deschampsia extract. Accordingly, in this disclosure Deschampsia extract means water extract unless otherwise indicated.

The HPLC analysis revealed similar pattern of flavonoid composition when water, methanol and EA were used, but its concentration decrease along with polarity. A very low amount of flavonoids were obtained in the n-hexane solvent.

In a further attempt to characterize the waste material obtained after aqueous extraction of Deschampsia plant, 50 g of dried byproduct was extracted with EA in the same way that fresh plant tissue. The extraction yield was 3.85% (wt-%). A dark green product was obtained, indicating a high chlorophyll extraction together with some non-polar compounds. This extract could not be dried which suggest the presence of oil substances. The HPLC analysis revealed that flavonoid compounds were absent in this fraction.

### Example 3

### Fractionation and Characterization of the Aqueous Extracts

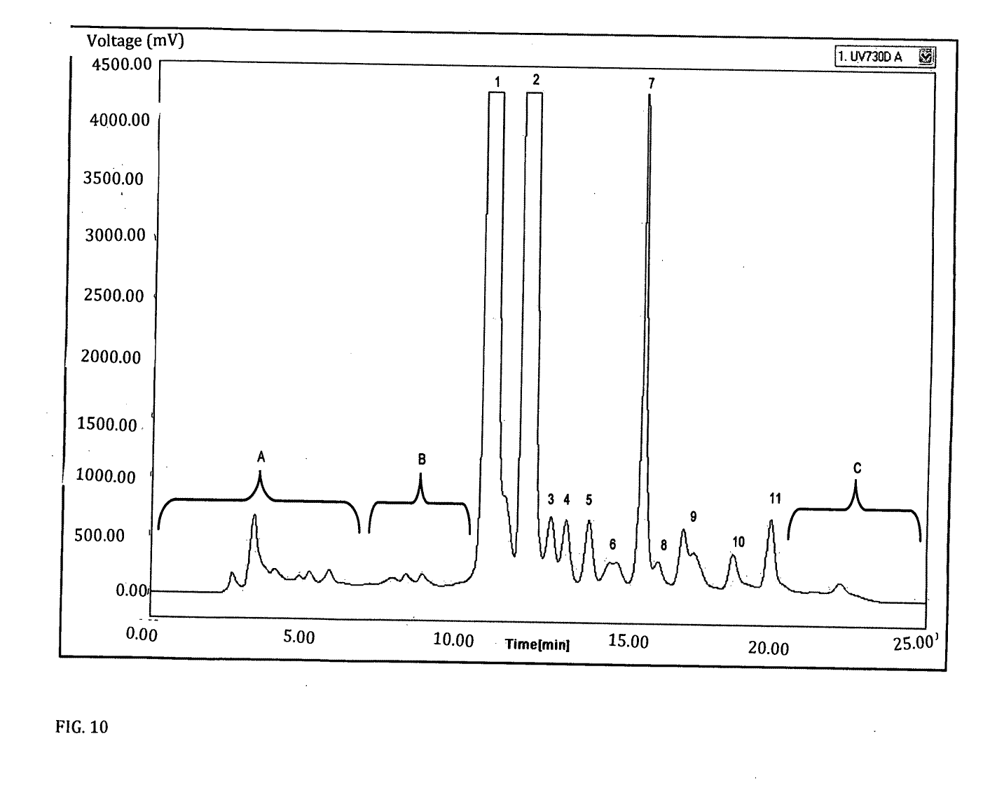

The aqueous extract of Deschampsia antarctica was analyzed with HPLC analysis. In order to elucidate the chemical structure of natural compounds from the extract the HPLC was coupled to an online mass spectrometer (MS) detector to obtain the molecular weight of individual peaks. FIG. 10 show the liquid chromatogram presenting the main components of Deschampsia antarcica aqueous extract. Small peaks that appear in the first region of the chromatogram are called fraction A and B, the tail fraction of the chromatogram is called fraction C.

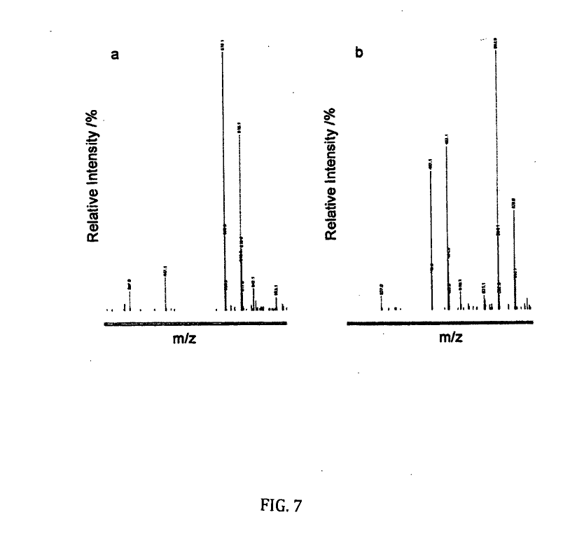

Both of the peaks were collected separately by using a semipreparative column. For purity assessment and HPLC_DAD equipped with an analytical column was used. The chromatograms corresponding to the isolated peaks 1 and 2 of FIG. 10 are presented in FIG. 7. Each chromatogram shows only one peak with purity higher than 95% indicating that the peaks were essentially pure.

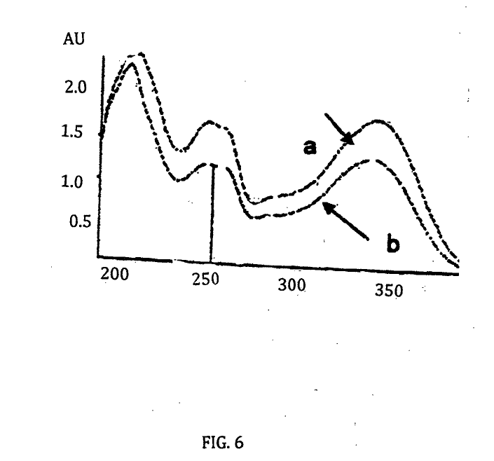

By using a Diode array analytical HPLC it was possible to obtain a UV-visible 200-400 nm spectrum for both of the compounds. The spectra are shown in FIG. 6.

Both spectrums showed major absorption bands in 240-400 nm region. Both exhibited first absorption band and 260 nm and second one at 350 nm. Those peaks are in close agreement with the absorption spectra exhibited by flavonoids when analyzed using UV-visible spectroscopy. When flavonoids are dissolved in methanol, flavones and flavonols show two major peaks known as band I (300-400 nm) and band II (240-280 nm), respectively.

In order to elucidate the chemical structures of the compounds, we coupled HPLC to mass spectrometer to obtain the mass chromatogram of individual peaks (FIG. 7 shows peaks 1 and 2). Based on these experiments, table 2 shows the results of Deschampsia extract analysis using HPLC-MS technique.

TABLE 2
Results from Deschampsia extract
analysis using HPLC-MS technique.

| Peak | RT | | | | |
|---|---|---|---|---|---|
| name | (min) | [M + H]⁺ | [M − H]⁻ | m/z | Identification |
| A | 2.04(+) | 705.26 | 703.41 | 704.4 | N.I. |
| A | 2.96(+) | 268.16 | 266.35 | 267.3 | N.I. |
| A | 3.44(+) | 927.26 | 925.47 | 926.7 | N.I. |
| A | 4.16(+) | 675.36 | 673.48 | 674.3 | N.I. |
| A | 5.36(+) | 659.37 | 657.55 | 658.6 | N.I. |
| A | 6.08(+) | 349.15 | 347.62 | 348.8 | N.I. |
| A | 7.04(+) | 583.30 | 581.56 | 582.9 | N.I. |
| B | 7.52(+) | 611.28 | 609.63 | 609.7 | 2″-O-β-L- |
| | 7.66(−) | | | | galactopyranosil |
| | | | | | orientin |
| B | 8.00(+) | 759.34 | 757.51 | 758.9 | N.I. |
| | 8.47(+) | 391.23 | 389.72 | 390.5 | N.I. |
| | 8.62(−) | | | | |
| 1 | 11.05(+) | 581.35 | 579.39 | 580.4 | 2″-O-β-O |
| | 11.24(−) | | | | arabinopyranoside |
| | | | | | orientin |
| | | | | | (orientin traces) |
| 2 | 12.28(+) | 595.38 | 593.54 | 594.8 | isowertiajaponine |
| | 12.35(−) | | | | (7-O-methyl |
| | | | | | orientin-2″-O- |
| | | | | | arabinopiranoside) |
| 3 | 12.78(+) | 595.35 | 593.72 | 594.7 | N.I. |
| | 12.81(−) | | | | |
| 4 | 13.51(+) | 637.38 | 635.63 | 636.7 | N.I. |
| | 13.52(−) | | | | |
| 5 | 14.25(+) | 579.35 | 577.88 | 576.5 | N.I. |
| | 14.18(−) | | | | |
| 6 | 15.20(+) | 679.3 | 677.76 | 678.9 | N.I. |
| | 15.12(−) | | | | |
| 7 | 13.51(+) | 637.38 | 635.58 | 636.6 | Isoswertiajaponin |
| | 16.06(−) | | | | 2″-O-β- |
| | | | | | arabinopyranoside |
| | | | | | acylated |
| 8 | 16.90(+) | 581.34 | 579.65 | | N.I. |
| | 16.90(−) | | | | |
| | 17.34(+) | 679.39 | 677.68 | 678.8 | N.I. |
| | 17.36(−) | | | | |
| 9 | 17.78(+) | 639.32 | 637.70 | 638.8 | N.I. |
| | 17.78(−) | | | | |
| 10 | 18.84(+) | 493.31 | 491.4 | 492.3 | N.I. |
| 10 | 19.26(+) | 563.38 | 561.7 | 563.0 | N.I. |
| 11 | 20.12(+) | 679.41 | 677.67 | 676.9 | N.I. |

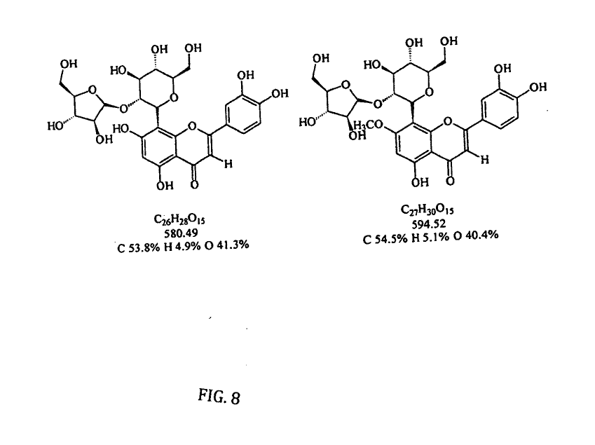

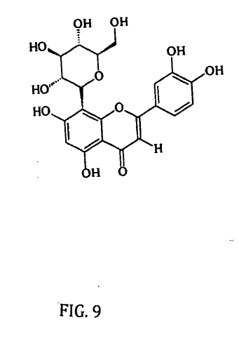

It can be observed that two major peaks (peaks 1 and 2) account 67% (w/w) of the total amount of injected sample. The mass spectra chromatogram showed the main peaks at m/z 580 and 480 for peak 1 and m/z 593 for peak 2. This information was compared with other mass spectra by using a mass spectra library for natural compounds and the resulting structures are presented in FIGS. 8 and 9. Peak 1 corresponds to isoswertiajaponin((7-O-methylorientin) 2″-O-beta arabinopyranoside) and orientine and peak 2 corresponds to orientin 2″-beta-arabinopyranoside. Pure orientine has never been reported to be present in Deschampsia extracts, but the other compounds have been previously indentified in Deschampsia antarctica leaves (Webby R. and Markham K, 1994, Isoswertijaponin 2″-O′beta-arabinopranoisee and other flavone-C glycosides from the Antarctica grass Deshampsia antarctica. Phytochemistry 36(5): 1323-1326).

In table 2 it is shown that in some cases one chromatographic peak was composed by a pool of compounds, e.g. peaks 6 and 10. Some of the compounds have not yet been indentified, but the m/z ratios are presented. Based on the gathered information, none of these unidentified compounds match with previously described flavonoids in Deschampsia antarctica extracts (Webby an Markham 1994). This fact demonstrates that we have identified novel flavonoids in the aqueous extract of Deschampsia.

We attempted to identify the unknown peaks by comparing the HPLC peaks with peaks with commercially available products. For this purpose the retention times of the peaks were compared. Of seven commercially available compounds that were previously described in Deschampsia antarctica, five corresponded to compounds that were in our extract. The correspondence was found with orientin and luteolin, phenolic acids, ferulic acid, cafeic acid and coumaric acid (results not shown). All of these, except orientin, have been previously identified in Deschampsia antarctica. None of the peaks of the seven commercially available compounds corresponded with peaks 6 and 10 (peaks that are identified as biologically most active in below).

To continue with the identification, the aqueous extract was passed through a CL-EM system that included an Agilent 1100 HPLC coupled to a Mass Spec electrospray Esquire 4000 ESI-IT. As a result the molecular mass, based on cations and anions, we identified the ion precursors of the following six compounds: 2-O-beta galactopyranosylorientin, 2″-O-beta arabinopyranoside orientin, orientin, isowetiajaponin (7-O-metilorientin 2″-O-Arabinopyranoside), Isowertijaponin (7-O-metilorientine 2″-)-Arabinopyranoside) and luteoline. None of these compounds either correspond with peaks 6 and 10.

### Example 4

### Biological Activity of Deschampsia-Extract

### Fractionation of Total Extracts

Compounds were separated through paper chromatography to extract GCB. The sample was seeded on Whatman No. 3 paper using 15% glacial acetic acid as the mobile phase. The different compounds were visualized under UV light. The different fractions called B1, B2 and B3, were recovered from the paper by immersion in methanol and then concentrated in a rotoevaporator. The slide chromatography was done to visualize the isolated compounds in each fraction.

Chemical Structure of the Fraction with Biological Activity

According to the results provided by the HPLC-mass spectrometry it can be assumed that luteolin with different degrees of glycosylation and substitution of glycosides through C—C bonds is the molecule that is largely present and causing biological activity. This type of structure increases the stability of the active compound. Moreover, these compounds are present in extracts of in vivo grown Antarctic Deschampsia plants or plants subjected to 4° C. for 72 hours, but they are not present in plants produced in vitro at 13° C. (data not shown). This indicates that they are compounds inducible at low temperatures or other types of stress the plants experience in wild.

It is known that flavones play an important role in the human body as an antioxidant, chelators of free radicals, anti-inflammatory agents, promoters of the metabolism of carbohydrates and stimulators of the immune system (Rahman, I., Biswas, S. K., Kirkham, P. A. 2006. Regulation of inflammation and redox signaling by dietary polyphenols. Biochem Pharmacol. 702(11): 1439-1452; Kandaswami, C. Lee, L. T. Lee P. P, Hwang J. J.; Ke. F. C., Huang, Y. T. Lee, M. T. 2005 In vivo 19(5) 895-909). However, there is no research or indications of Deschampsia antactica extracts of being antineoplastic.

In order to determine whether the methanol extracts obtained from D. antarctica could have some antineoplastic effect, the soluble fractions B1, B2 and B3 were tested. These fractions were obtained from the total fraction and have different degrees of glucoside substitution.

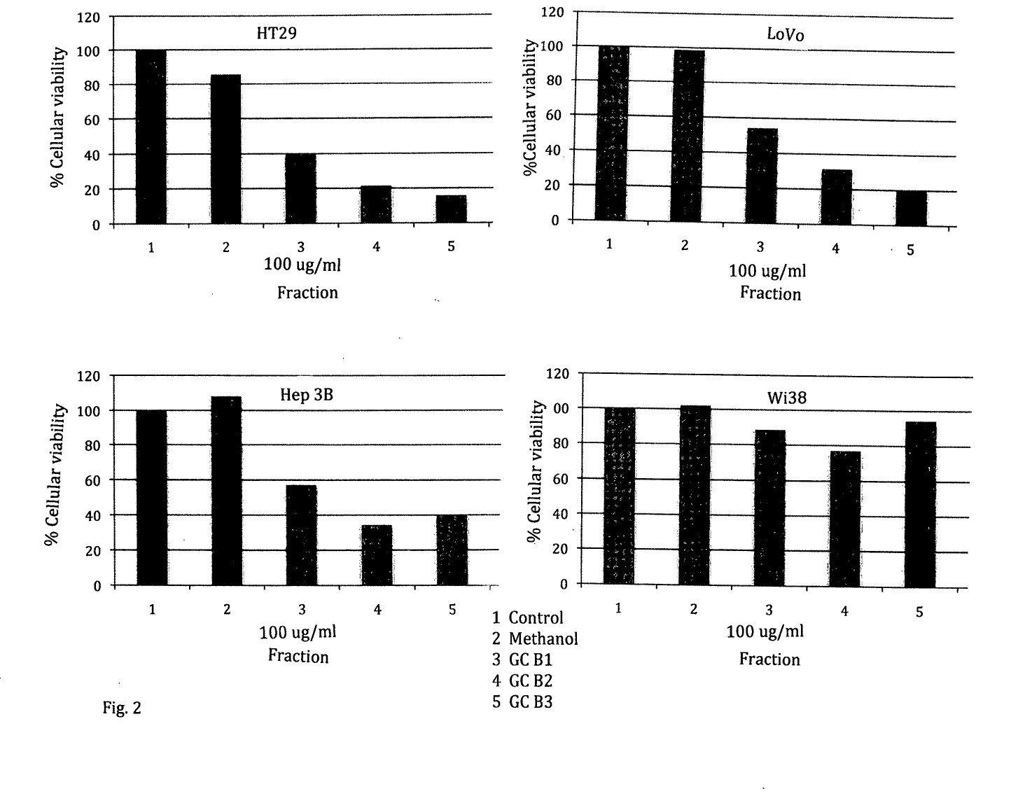

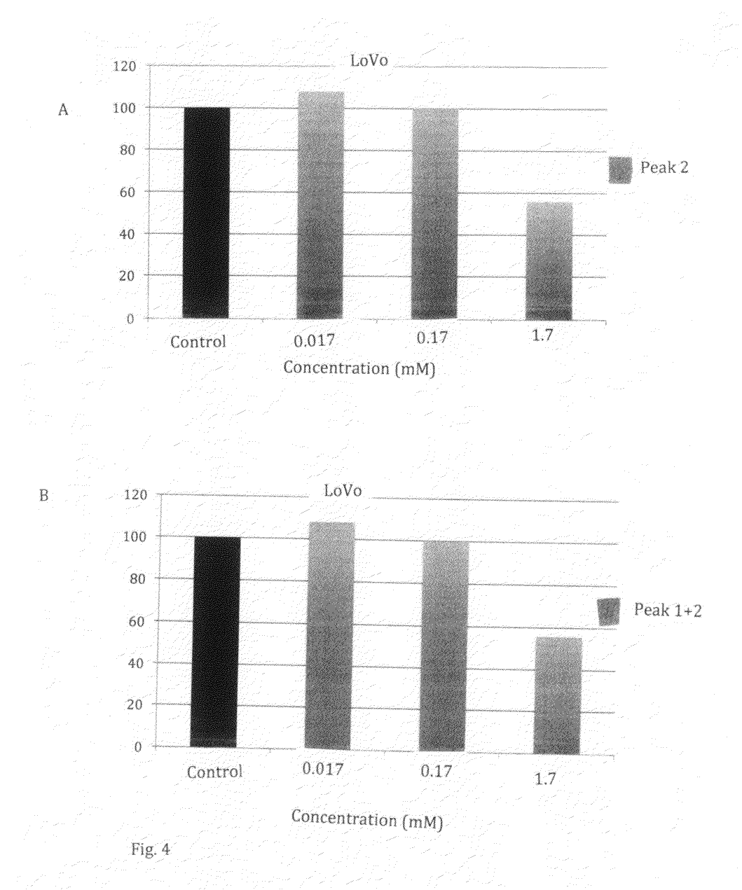

FIG. 2 shows the effect of the B1, B2 and B3 fractions on in vitro growth of colon cancer cells and hepatic cancer cells. It can be seen that these fractions effectively inhibit the proliferation of human HT 29 and LoVo colorectal cancer cells and Hep3B hepatoma cancer cells while the B3 fraction, with the highest degree of glucoside substitution, presents the greatest level of inhibition on malignant cellular proliferation (FIG. 4 HT29, LoVo and Hep 3B). Its effect on WI38 (normal king fibroblasts) was tested at the maximum concentration to determine specificity and toxicity. No inhibitory effect on WI38 cells proliferation was observed (FIG. 2).

It can be concluded that these compounds can inhibit malignant cells growth in vitro, but showed no inhibitory effect on the proliferation of normal fibroblasts. These data indicate the potential antineoplastic effect of these fractions.

As shown in Examples above, the antineoplastic compounds extracted from Deschampsia antarctica could be induced in vitro. Therefore, the amount of antioxidants to be produced in plants by exposure to UV light, salt treatment or low temperature can be modulated. Moreover, the production of the antineoplastic extract becomes actually practicable, as the plat material can be cultivated in large amounts in vitro.

Besides working with the soluble fractions mentioned before, Deschampsia antarctica plant material was extracted with solvents of increasing polarity (ethyl acetate and methanol). The aim of this approach was to divide plant constituents into fractions of different polarity on extraction. Organic solvent extracts were made in a Soxhlet apparatus.

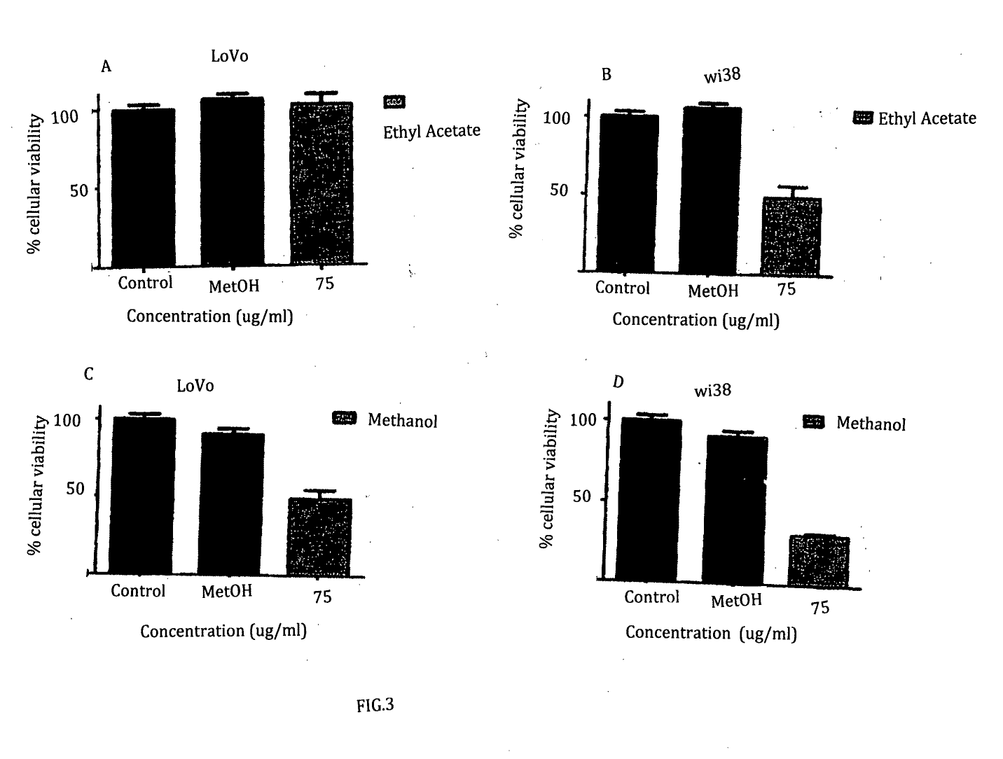

FIG. 3a shows the effect of an ethyl acetate extract on in vitro growth of human colon cancer cells (LoVo). An inhibition of 50% was observed in the cellular proliferation of these tumoral cells. The same extract was tested in WI 38 cells (normal lung fibroblasts). Non-inhibitory effect on WI 38 cells proliferation was observed (3b).

On the other hand, FIG. 3c shows the effect of a methanol extract, which produced more than 50% of inhibition on the proliferation of colon tumoral cells (LoVo). This extract was tested in non-tumoral cells (WI38), showing an inhibitory effect on cell proliferation (FIG. 3d). The methanol and ethyl acetic extracts were active against LoVo colorectal cancer cells at the lowest concentration of 75 ug/ml.

The most active fractions were used for further fractionation steps. This procedure led to the isolation of pure compounds (see Example 3 above). We also tested these pure compounds (peak 1 and peak 2 of example 3 above) and a combination of them on tumoral and non-tumoral cells.

FIGS. 4a and 4b show the inhibitory effect of pure compounds, alone an in combination (peak 2 and the combination of peak 1 and peak 2) on colon cancer cells (LoVo). These compounds were isolated from Deschampsia antarctica extracts as described in Example 3 above.

The inhibitory effect of cellular proliferation was observed with peak 2 and with a combination of peak 1 and 2 at concentrations of 1.7 mM, which corresponds to 1000 μg/ml, this concentration being 10 time higher than the inhibitory concentration of the ethyl acetate and methanol extracts. This result proves that methanol and ethyl acetate extracts of Deschampsia antarctica are efficient in 10 time lower concentration than the purified compound.

### Example 5

### Biological Activity of Individual Peaks of the Extract

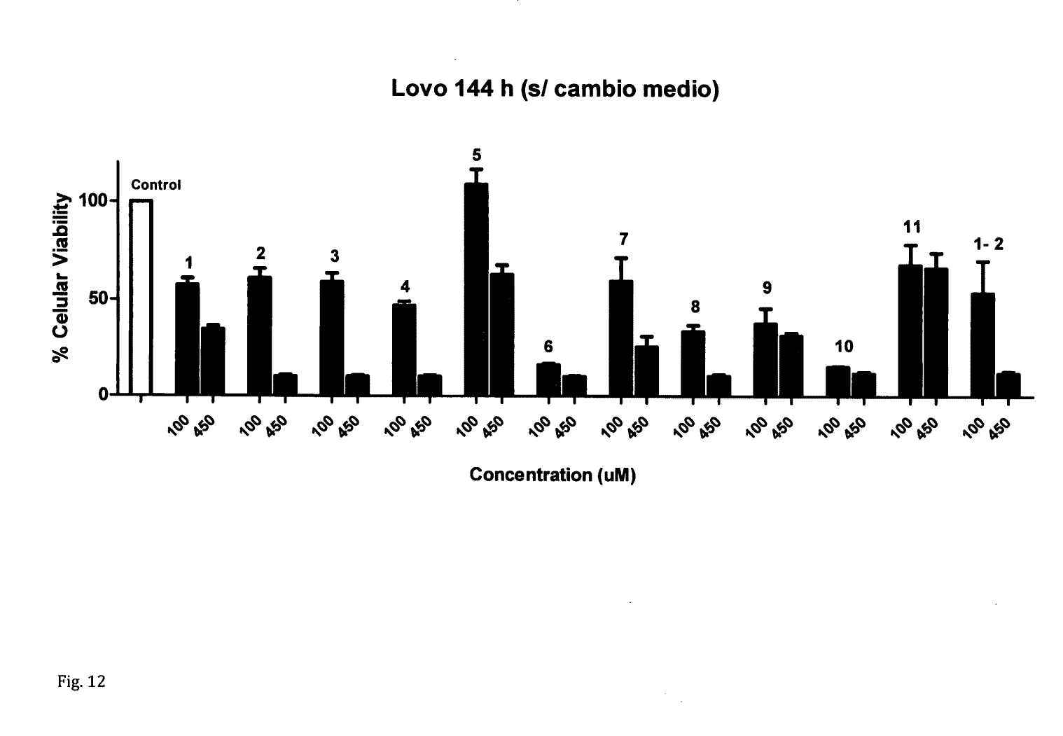

An assay to test the biological activity of individual peaks was carried out using the same antineoplastic test already evaluated for the whole extract. The main purpose of these studies was to identify who are the peaks with the highest biological activity and to determine a synergistic effect among the different compounds. The data is presented in FIG. 12.

It could be observed than all peaks exert close to 50% inhibition of the tumor cell proliferation at a putative concentration of 450 uM, except for those compounds present in peak 5. However, at a lower concentration (100 uM) only peaks 6, 8, 9 and 10 exhibit a certain degree of inhibition of tumor cells proliferation, peaks 6 and 10 being the most powerful inhibitors of colorectal cancer cell proliferation.

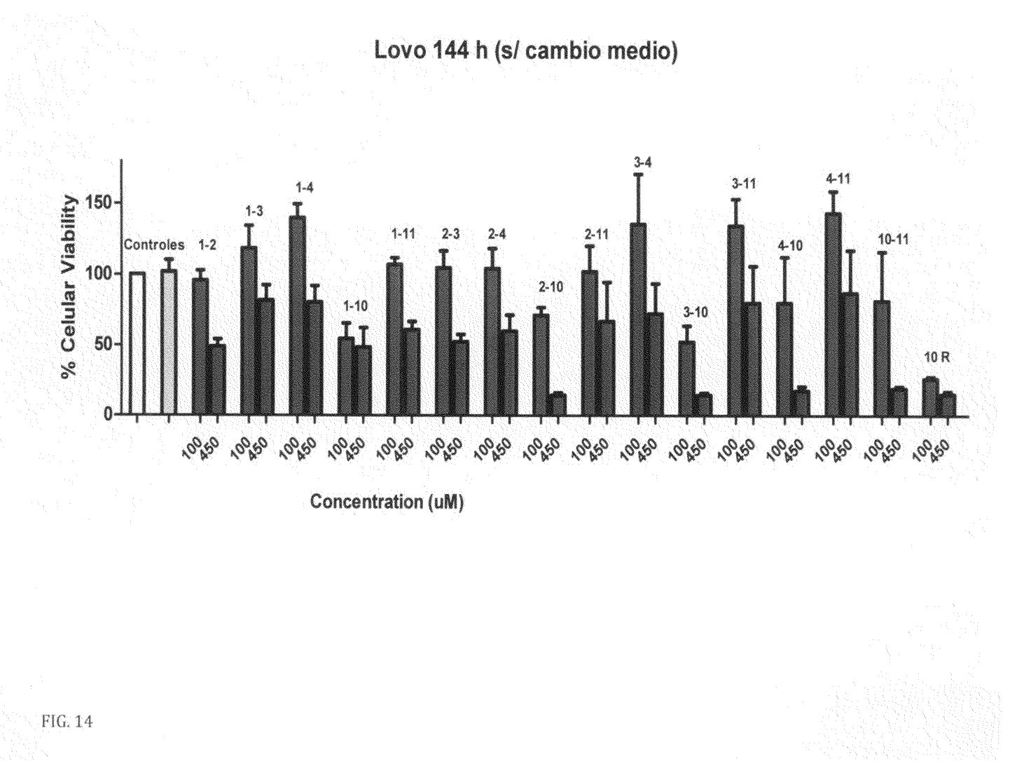

A further assay was designed to test the synergistic activities of the compounds of the peaks. Results are shown in FIG. 14. It can be seen that each combination, containing peak 10 as one of its components, showed the highest antineoplastic activity at 450 uM. Thus peaks 1 and 2 identified as orientin and derivatives showed only a weak antineoplastic activity in the same concentration. No synergistic effect was found among tested peaks.

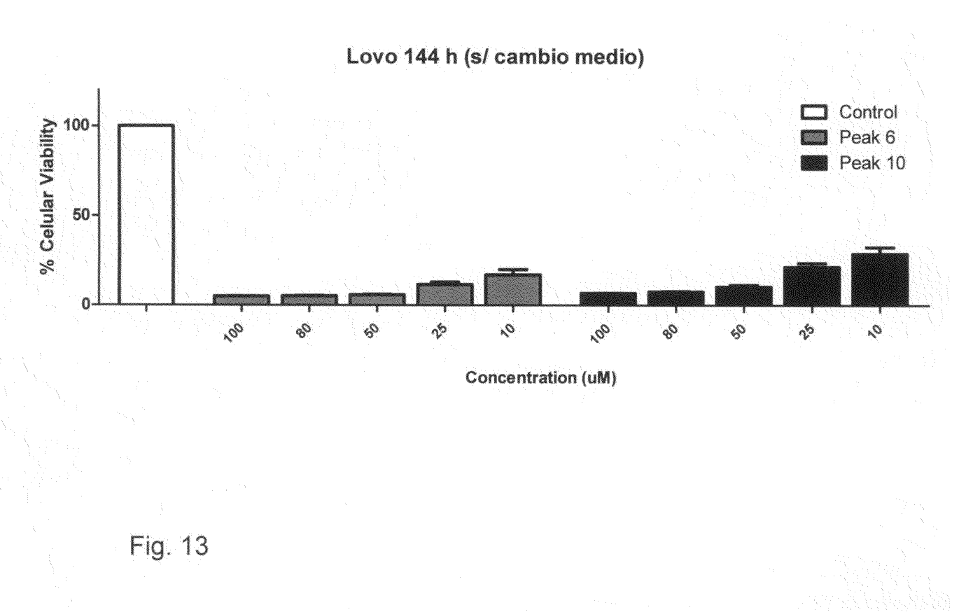

In order to test the antineoplastic activity of the most active peaks (6 and 10) an assay using Lovo-cells was carried out and results are presented in FIG. 13. It was clear that peak 6 was more active than 10 because it showed higher inhibition of Lovo cells at lower concentration (10 uM). Thus the present example is the first report concerning the biological activity of these natural compounds present in peak 6 an d10, respectively, which in turn are the responsible for the higher antineoplastic activity of the whole extract.

Thus, the present example is the first report concerning the biological activity of these new natural compounds present in peak 6 and 10, respectively. These still unidentified peaks appear to be responsible for the higher antineoplastic activity of the whole extract.

### Example 6

### Preparation of Fast-Dissolving Tablets for Oral Administration Comprising 500 mg of Deschampsia antarctica Extract

We provide here a composition for oral administration of the Deschampsia antarctica extract for prophylacic, preventive and curing purposes for patients suffering or prone to cancerous and tumoral diseases. Tablets each exhibiting the following qualitative and quantitative composition:

Deschampsia antarctica Luteoline extract 500 mg, D-glucosa monohydrate 597.6 mg, Sodium croscarmellose 35.2 mg, Microcrystalline cellulose 160.0 mg, Anhydrous citric acid 35.2 mg, Granulated sorbitol 160.0 mg, Aspartame 28.8 mg, Saccharin sodium 14.4 mg, Glycerol dibehenate 16.0 mg, Magnesium stearate 6.4 mg, Orange flavoring 46.4 mg, are prepared in the following way: all the components, with the exception of lubricating agents (magnesium stearate and glycerol dibehenate), are mixed by means of a tumbler until a homogeneous whole is obtained, the magnesium stearate and glycerol dibehenate are added and mixing is again carried out until homogeneous, then the resulting mixture is subjected to tableting in order to obtain tablets exhibiting a unit weight 1.6 g which measure 20 mm in diameter and 4.5 in height. The tablets thus prepared disintegrate in the mouth in 30 seconds.

### Example 7

### Preparation of Fast-Dissolving Tablets Comprising 7.5 mg of Deschampsia antarctica Extract

Tablets exhibiting the following qualitative and quantitative composition for 100 g:

Ingredients Quantity: Deschampsia antarctica Luteoline extract 7.5 g, Spray-dried mannitol 71.0 g, Microcrystalline cellulose 15.0 g, Sodium croscarmellose 3.0 g, Ammonium glycyrrhizinate 0.3 g, Aspartame 1.0 g, L-menthol 0.2 g, Mint flavouring 1.0 g, Magnesium stearate 1.0 g are prepared in the following way: all the components, with the exception of Magnesium stearate, are mixed by a tumbler until a homogeneous whole is obtained, the Magnesium stearate is added and mixing again carried out until homogenous, then the mixture is subjected to tableting. The tablets thus prepared disintegrate in the mouth in 20 seconds.

### Example 8

### Preparation of Pellets Containing Deschampsia antarctica Extract

900 g of Deschampsia antarctica extract, 800 g of microcrystalline cellulose, 12 g of colloidal silicon dioxide, 684 g of sodium chloride and 36 g of potassium chloride were mixed. The mixture was transferred to a fluidization rotogranulator, and a mixture of 40 g of 35% dimethyl polysiloxane emulsion and 2000 ml of ion-exchanged water was sprayed onto it. Spraying speed of the pelletizing liquid was set at 50 ml/min, pressure of the spraying air was 2.5 bar. The speed of the rotor was set at 450 rev/min in the first 15 minutes of the pelletization and later kept at 600 rev/min. Speed by volume of the fluidization air was kept at 60 m3/hour in the first 15 minutes of the pelletization and later at 90 m3/hour. The temperature of the fluidization air was set at 25° C. in the first part of the pelletization and 40° C. for the drying procedure. The dried pellets were passed through sieves 1.6 mm.

What is claimed is:

1. An antineoplastic extract, prepared from Deschampsia antarctica plants, where biologically active group of compounds can be identified by HPLC at retention time 15.12-15.20 min, and m/z of 678.9; at retention time 18.84 min, and m/z of 492.3, and at retention time 19.26 min, and m/z of 563.0.

2. The extract of claim 1, wherein the plants are grown in vitro.

3. An antineoplastic composition purified from an extract prepared from Deschampsia antarctica, where the composition comprises biologically active compounds that can be identified by HPLC at retention time 15.12-15.20 min, and m/z of 678.9; at retention time 18.84 min, and m/z of 492.3, and at retention time 19.26 min, and m/z of 563.0.

4. The antineoplastic composition of claim 3, wherein the extract is prepared from in vitro grown Deschampsia antarctica plants.

5. A method to prevent proliferation of cancer cells, said method comprising treating patients with a composition purified from an extract prepared from Deschampsia antarctica, where the composition comprises biologically active compounds that can be identified by HPLC at retention time 15.12-15.20 min, and m/z of 678.9; at retention time 18.84 min, and m/z of 492.3, and at retention time 19.26 min, and m/z of 563.0.

6. The method of claim 5, wherein the cancer cells are colon cancer cells.
